(19) United States
(12) Patent Application Publication
(10) Pub. No.: US 2014/0275893 A1
(43) Pub. Date: Sep. 18, 2014

(54) Method and System for Positioning a Sensor

(71) Applicant: COVIDIEN LP, Mansfield, MA (US)

(72) Inventor: Jeffrey A. Booker, Boulder, CO (US)

(73) Assignee: COVIDIEN LP, Mansfield, MA (US)

(21) Appl. No.: 13/829,042

(22) Filed: Mar. 14, 2013

(51) Int. Cl. A61B 5/00; A61B 5/1455

(52) U.S. Cl. CPC A61B 5/684; A61B 5/14552
USPC 600/340; 600/372; 600/383; 29/428

(57) ABSTRACT

Release liners configured to facilitate the positioning of a sensor at a predetermined position of a patient are provided. The release liners may include one or more alignment features configured to approximately align with one or more anatomical features of the patient when the sensor is placed in the predetermined position. The one or more alignment features may include any suitable number of alignment lines. The release liner may be at least partially removable from the sensor.

### BACKGROUND

The present disclosure relates generally to medical devices, and more particularly, to sensors used for sensing physiological parameters of a patient.

This section is intended to introduce the reader to various aspects of art that may be related to various aspects of the present disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

In the field of medicine, doctors often desire to monitor certain physiological characteristics of their patients. Accordingly, a wide variety of devices have been developed for monitoring certain physiological characteristics of a patient. Such devices provide doctors and other healthcare personnel with the information they need to provide the best possible healthcare for their patients. As a result, such monitoring devices have become an indispensable part of modern medicine.

One technique for monitoring certain physiological characteristics of a patient is commonly referred to as pulse oximetry, and the devices built based upon pulse oximetry techniques are commonly referred to as pulse oximeters. Pulse oximetry may be used to measure various blood flow characteristics, such as the blood-oxygen saturation of hemoglobin in arterial blood, the volume of individual blood pulsations supplying the tissue, and/or the rate of blood pulsations corresponding to each heartbeat of a patient.

Another technique for monitoring physiological characteristics of a patient is commonly referred to as electroencephalography (EEG), and the devices built based upon electroencephalographic techniques are commonly referred to as EEG monitors. EEG monitors use non-invasive electrophysiological monitoring to evaluate global changes in a patient's condition, for example, during surgical procedures. Examples of global changes may include assessing the effects of anesthetics, evaluating asymmetric activity between the left and right hemispheres of the brain in order to detect cerebral ischemia, and detecting burst suppression. One such technique includes bispectral index (BIS) monitoring to measure the level of consciousness by algorithmic analysis of a patient's EEG during general anesthesia.

Often the monitoring devices, or probes or sensors associated with the monitoring devices, are applied to the patient. For example, sensors for use with pulse oximetry monitors may be applied to a blood perfused tissue of the patient, such as the forehead. Additionally, electrodes for use with EEG monitors may be applied to the temple and forehead of the patient. Proper placement of the sensing components (e.g., the optical components or the electrodes) of the sensor relative to the patient helps to correctly calculate the physiological characteristics (e.g., blood oxygen saturation, heart rate, or BIS). Misplacement of the sensing components may increase the algorithmic work, filtering, and artifacting to obtain the physiological characteristics.

### BRIEF DESCRIPTION OF THE DRAWINGS

Advantages of the disclosed techniques may become apparent upon reading the following detailed description and upon reference to the drawings in which:

FIG. 1 is a perspective view of a patient monitoring system configured to monitor one or more physiological parameters of a patient, in accordance with an embodiment;

FIG. 2 is a diagram of a forehead pulse oximetry sensor including a release liner having alignment features, in accordance with an embodiment;

FIG. 3 is a diagram of a forehead pulse oximetry sensor including a release liner having alignment features, in accordance with an embodiment;

FIG. 4 is a diagram of a BIS sensor including a release liner having alignment features, in accordance with an embodiment;

FIG. 5 is a diagram of a BIS sensor including a release liner having alignment features, in accordance with an embodiment;

FIG. 6 is process flow diagram of a method for manufacturing a sensor including a release liner having alignment features, in accordance with an embodiment; and

FIG. 7 is a process flow diagram for remanufacturing a sensor including a release liner having alignment features, in accordance with an embodiment.

### DETAILED DESCRIPTION OF THE SPECIFIC EMBODIMENTS

One or more specific embodiments of the present techniques will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

As noted above, sensors associated with monitoring devices may be applied to a patient to monitor physiological parameters of the patient. Misplacement of the sensing components of a sensor on the patient, which may result from misplacing the sensor on the patient, may increase the algorithmic work, filtering, and artifacting to obtain the physiological parameters. Unfortunately, caregivers may experience difficulty in determining a desired placement of a sensor and/or in properly positioning the sensing components of sensor on a tissue of the patient.

The present disclosure is generally directed to sensors including features to facilitate the proper placement of the sensors on a patient's tissue. For example, a sensor may include a release liner that includes alignment features (e.g., lines, indicia, shapes, labels, text, arrows, etc) to facilitate the proper placement of a sensor on a patient. In certain embodiments, the release liner may include a larger surface area than a sensor body of the sensor. That is, at least a portion of the release liner may extend past the sensor body. In certain embodiments, the release liner may be transparent such that anatomical features of the patient may be viewable through the release liner as the sensor is applied to the patient. In this manner, a caregiver may align an alignment feature over an anatomical feature of the patient to position the sensor in a desired position. Additionally, the release liner may be removably attached to a sensor body of the sensor. Thus, a caregiver may remove the release liner after positioning the sensor on the patient to increase patient comfort. It should be noted that while certain embodiments of the present disclosure are discussed in the context of pulse oximetry sensors and BIS sensors, the embodiments disclosed herein may be implemented on any suitable medical sensor, such as regional oximetry sensors, sensors for measuring water fraction, temperature sensors, electrocardiograph sensors, acoustic sensors, impedance sensors, or the like.

With the foregoing in mind, FIG. 1 illustrates an embodiment of a patient monitoring system 10 that may include a patient monitor 12 and a sensor 14, such as a forehead pulse oximetry sensor, to monitor physiological parameters of a patient. By way of example, the sensor 14 may represent a MAXFAST™, NEOMAX™, or other pulse oximetry sensor available from Nellcor Puritan Bennett, LLC. Although the depicted embodiments relate to sensors for use on a patient's forehead and/or temple, it should be understood that, in certain embodiments, the features of the sensor 14 as provided herein may be incorporated into sensors for use on other tissue locations, such as the finger, the toes, the heel, the ear, stomach, chest, back, or any other appropriate measurement site. In addition, although the embodiment of the patient monitoring system 10 illustrated in FIG. 1 relates to photoplethysmography or pulse oximetry, the patient monitoring system 10 may be configured to obtain a variety of medical measurements with a suitable medical sensor. For example, the patient monitoring system 10 may additionally or alternatively be configured to perform regional oximetry, determine patient electroencephalography (e.g., a bispectral (BIS) index), or any other physiological parameter such as tissue water fraction or hematocrit.

The sensor 14 may include one or more emitters 16 and one or more detectors 18 to acquire a physiological signal corresponding to one or more physiological parameters of a patient. For pulse oximetry applications, the emitter 16 may transmit at least two wavelengths of light (e.g., RED light and/or IR light) into a tissue of the patient. For example, the RED light may have a wavelength of about 600 nm to 700 nm, and the IR light may have a wavelength of about 800 nm to 1000 nm. In other applications, a tissue water fraction (or other body fluid related metric) or a concentration of one or more biochemical components in an aqueous environment may be measured. As such, the emitter 16 may transmit two or more wavelengths of light, most commonly near infrared wavelengths between about 1,000 nm to about 2,500 nm. However, any appropriate wavelength (e.g., green, yellow, etc.) and/or any number of wavelengths (e.g., one or more) may be used. The detector 18 may be a photodetector selected to receive light in the range emitted from the emitter 18 after it has passed through the tissue. Additionally, the emitter 16 and the detector 18 may operate in various modes (e.g., reflectance or transmission). However, as noted above, the patient monitoring system 10 may be configured to determine a variety of physiological parameters with a suitable medical sensor. Accordingly, in certain embodiments, the sensor 14 may include sensing components in addition to, or instead of, the emitter 16 and the detector 18. For example, in one embodiment, the sensor 14 may include one or more electrodes (e.g., four electrodes) to obtain an electroencephalography signal.

The sensor 14 may be communicatively coupled to the patient monitor 12. In certain embodiments, the emitters 16 and detectors 18 of the sensor 14 may be coupled to the patient monitor 12 via a cable 20 through a plug 22 (e.g., a connector having one or more conductors) coupled to a sensor port. However, in other embodiments, the sensor 14 may include a wireless module configured to establish a wireless communication with the patient monitor 12 using any suitable wireless standard. The patient monitor 12 may be configured to calculate physiological parameters of the patient relating to the physiological signal received from the sensor 14. For example, the patient monitor 12 may include a processor configured to calculate the patient's arterial blood oxygen saturation, pulse rate, a bispectral index, and/or any other suitable physiological characteristics. Additionally, the patient monitor 12 may include a monitor display 26 configured to display information regarding the physiological parameters, information about the system (e.g., instructions for placement of the sensor 14), and/or alarm indications. The patient monitor 12 may include various input components 28, such as knobs, switches, keys and keypads, buttons, etc., to provide for operation and configuration of the patient monitor 12.

As noted above, the patient monitor 12 may be any suitable monitor, such as a pulse oximetry monitor or an electroencephalography monitor. Furthermore, to upgrade the conventional operation provided by the patient monitor 12 and to provide additional functions, the patient monitor 12 may be coupled to a multi-parameter monitor 30 via a cable 32 connected to a sensor input port or via a cable 34 connected to a digital communication port. In addition to the patient monitor 12, or alternatively, the multi-parameter monitor 30 may be configured to calculate physiological parameters and to provide a central display 36 for the visualization of information from the patient monitor 12 and from other medical monitoring devices or systems. The multi-parameter monitor 30 includes a processor that may be configured to execute code for calculating one or more physiological parameters and displaying the physiological parameters and/or other information about the patient monitoring system 10. The multi-parameter monitor 30 may also include various input components 38, such as knobs, switches, keys and keypads, buttons, etc., to provide for operation and configuration of the multi-parameter monitor 30. In addition, the patient monitor 12 and/or the multi-parameter monitor 30 may be connected to a network to enable to the sharing of information with servers or other workstations.

The sensor 14, illustrated as operatively connected to the patient monitor 12, may include a sensor body 40 to house the sensing components of the sensor 14 (e.g., the emitter 16 and the detector 18). The sensor body 40 may be formed from any suitable material, including rigid or conformable materials, such as fabric, paper, rubber, or elastomeric compositions (including acrylic elastomers, polyimide, silicone rubber, celluloid, PMDS elastomer, polyurethane, polypropylene, acrylics, nitrile, PVC films, acetates, and latex). In certain embodiments, the sensor body 40 may include an adhesive layer disposed on a patient-contacting surface of the sensor body 40 to attach the sensor 14 to the patient's tissue.

The sensor 14 may include a release liner, which may be removably attached to the sensor body 40 (e.g., opposite the patient-contacting surface). In certain embodiments, the release liner may include alignment features to facilitate the positioning of the sensor 14 on a desired tissue location of the patient. It should be noted that the alignment features, as described below, may be suitable for use with an adult patient, a child patient, and/or a neonatal patient. Further, while the embodiments described below may relate to alignment features configured to position the sensor 14 on a forehead of the patient, it is also contemplated that alignment features may be included on the sensor 14 that are configured to facilitate the positioning of the sensor 14 relative to any anatomical feature of the patient (e.g., the midline of the chest, the navel, and/or the collarbone).

For example, FIG. 2 illustrates an embodiment of the sensor 14 including a release liner 50. As illustrated, the sensor 14 may be a pulse oximetry sensor that is applied to a forehead 52 of a patient. Accordingly, the release liner 50 may include one or more alignment features 54 to facilitate the positioning of the sensor 14 on the forehead 52. As will be appreciated, the alignment features 54 may be specific for a desired tissue location (e.g., the forehead, chest, stomach, etc.) and/or for a sensor type (e.g., a pulse oximetry sensor, a BIS sensor, a regional oximetry sensor, etc.). The alignment features 54 may include any suitable features to facilitate the positioning of the sensor 14 relative to the patient, such as lines, geometric shapes (e.g., an eye, an eyebrow, a circle, an oval, a rectangle, etc.), arrows, text, numbers, symbols, measurement lines, colors, or the like. Additionally, the release liners 50 may be formed of a transparent or semi-transparent material. For example, the release liner 50 may be constructed from papers, fabrics, Kraft papers, plastic films (e.g., biaxially-oriented polyethylene terephthalate, biaxially-oriented propylene, polyolefins, etc.), or any other suitable material. The transparency may enable a caregiver to view anatomical features of the patient through the release liner 50, thereby facilitating the alignment of the alignment features 54 with their respective anatomical features. However, in other embodiments, the release liner 50 may be formed of any suitable material (e.g., transparent, semi-transparent, or opaque), and the one or more alignment features 54 may include cut-outs (e.g., holes). In this manner, the caregiver may view anatomical features of the patient through the cut-outs of the release liner 50 to facilitate the alignment of the alignment features 54 with their respective anatomical features.

Additionally, the release liner 50 may be removably attached to the sensor body 40. In certain embodiments, the release liner 50 may be removably attached to the sensor body 40 (e.g., a non-patient-contacting surface of the sensor body) via an adhesive, such as an acrylic-based adhesive, a hydrocolloid adhesive, a supported transfer tape, an unsupported transfer tape, or any combination thereof. Accordingly, the release liner 50 may be removed from the sensor body 40 after the sensor 14 is placed at a desired position on the patient. In embodiments in which the release liner 50 is removably attached via an adhesive, portions of the sensor body 40 may include a residue of the adhesive after the release liner 50 is removed. Accordingly, in some embodiments, one or more covers (not shown) may be provided (e.g., in the sensor packaging or in a sensor kit) that may be placed over the portions of the sensor body 40 having an adhesive after the release liner 50 is removed. In this manner, the covers may reduce the transfer of any remaining adhesive on the non-patient-contacting surface of the sensor body 40 from the non-patient contacting surface to an undesired location (e.g., the caregiver, the patient, etc.). The one or more covers may be constructed from papers, fabrics, Kraft papers, plastic films (e.g., biaxially-oriented polyethylene terephthalate, biaxially-oriented propylene, polyolefins, etc.), or any other suitable material, and the covers may include an adhesive to facilitate securing the cover on the sensor body 40. However, in other embodiments, the release liner 50 may be configured to separate from the sensor body 40 via perforations along the release liner 50. That is, the caregiver may pull the release liner 50, and the release liner 50 may separate along a perforated line. Thus, in embodiments in which the release liner 50 is removable attached via perforations, at least a portion of the release liner 50 may be present on the sensor 14 after another portion of the release liner 50 is removed.

As noted above, it may be desirable to provide alignment features 54 that are specific for the sensor type and/or the desired tissue location. For example, in embodiments in which the sensor 14 is a forehead pulse oximetry sensor, it may be desirable to position the sensor 14 above an eyebrow 56 of the patient and to position the sensing components (e.g., the emitter 16 and the detector 18) of the sensor 14 lateral of a pupil 58 of the patient and proximal to (e.g., near) the temple 60 of the patient. Accordingly, the release liner 50 may extend past a lower edge 62 of the sensor body 40, such that the release liner 50 may cover the eyebrow 56 and/or the pupil 58 of the patient when the sensor 14 is applied to a desired position on the forehead 52. The alignment features 54 may include a first alignment line 64 that is configured to approximately align with the eyebrow 56 when the sensor 14 is placed in the desired position. That is, the first alignment line 64 may be located on the release liner 50 in an expected location of the eyebrow 56 when the sensor 14 is properly applied. As illustrated, the first alignment line 56 may be a horizontal line. However, in other embodiments, the first alignment line 56 may include one or more arches (e.g., curved portions), may vary in thickness, and/or may include an outline of an eyebrow shape. Additionally, in certain embodiments, the first alignment line 56 may include a label (e.g., “eyebrow”).

The alignment features 54 may include a second alignment line 66 to facilitate the positioning of the emitter 16 and the detector 18 of the sensor 14 relative to one or more anatomical features of the patient. The second alignment line 66 may be located on the release liner 50 in an expected location of the pupil 58 when the sensor 14 is applied to the forehead 52. Furthermore, the second alignment line 66 may be located on the release liner 50 such that the emitter 16 and detector 18 may be disposed on the forehead 52 lateral of the pupil 58 and proximal to the temple 60. As illustrated, the second alignment line 66 may be a vertical line that intersects (e.g., bisects) the pupil 58 when the sensor 14 is applied. It should be noted that, as described herein, alignment lines that are configured to be aligned with the pupil 58 are configured to be aligned with the pupil 58 when the patient is looking forward (i.e., the pupil 58 is approximately centered in the eye). In other embodiments, the second alignment line 66 may be a horizontal line that intersects the pupil 58, or may include any other suitable line or combination of lines (e.g., lines that form an “x”). Additionally, similar to the first alignment line 56, the second alignment line 66 may include a label (e.g., “pupil” or “eye”).

As noted above, the alignment features 54 may include geometric shapes to further facilitate the positioning of the sensor 14. In particular, the alignment features 54 may include geometric shapes corresponding to anatomical features of the patient, such as an eyebrow shape and/or an eye shape. This may be desirable to provide additional information to the caregiver regarding which of the anatomical features corresponds to a particular alignment feature 54. For example, FIG. 3 illustrates an embodiment of the sensor 14 and the release liner 50 having a first alignment shape 80. As illustrated, the first alignment shape 80 may be eye-shaped. In particular, the first alignment shape 80 may include lines located on the release liner 50 in an expected location of an eye 82 of the patient when the sensor 14 is applied to the forehead 52 in a desired position. As illustrated, the first alignment shape 80 may include dashed lines to enable the caregiver to more readily align the first alignment shape 80 with the eye 82 and/or the pupil 58. However, the first alignment shape 80 may be formed of any suitable lines or line type. Further, while the illustrated embodiment of the first alignment shape 80 includes lines configured to be aligned with the pupil 58, in other embodiments, the first alignment shape 80 may only include lines configured to be aligned with an outer portion of the eye 82. Additionally, it should be noted that the release liner 50 may include alternative or additional alignment shapes. For example, the release liner 50 may include a second alignment shape (not shown) that may be eyebrow-shaped.

While certain disclosed embodiments include release liners 50 and alignment features 54 to facilitate the positioning of sensors 14 including optical components configured for pulse oximetry, it is also contemplated that release liners 50 and alignment features 54 may be utilized to facilitate the positioning of sensors 14 configured to perform BIS measurements. Accordingly, FIG. 4 illustrates an embodiment of the sensor 14 including one or more electrodes 100 (e.g., electrode 100A, 100B, 100C, and 100D) and the release liner 50. The alignment features 54 may be configured to facilitate the positioning of the electrodes 100 on the forehead 52 in a desired position. In particular, it may be desirable to position the electrode 100A on the center of the forehead 52. Accordingly, the alignment features 54 may include a first alignment line 102 that is configured to approximately align with a vertical midline of the nose 104 when the sensor 14 is placed on the forehead 52 in the desired position. In other embodiments, the alignment features 54 may additionally or alternatively include a geometric shape corresponding to the nose 104. The release liner 50 may extend past the lower edge 62 of the sensor body 40, such that the release liner 50 may cover a portion or the entirety of the nose 104.

The release liner 50 may include additional alignment features 54 to facilitate the positioning of the other electrodes 100 (e.g., 100B, 100C, and 100D). For example, it may be desirable to position the electrode 100C above the eyebrow 56 and approximately in line with the pupil 58. Accordingly, the alignment features 54 may include a second alignment line 106 that may be configured to approximately align with the eyebrow 56 of the patient when the sensor 14 is applied in the desired position. Additionally, the alignment features 54 may include a third alignment line 108 that may be configured to approximately align with the pupil 58. That is, some variation in the location of the pupil 58 (and other anatomical features) is expected among patients, and as such, the alignment features 54 (e.g., the third alignment line 108) may be configured to align with an expected location of the pupil 58. The second alignment line 106 and the third alignment line 108 may be presented in a similar manner as the first alignment line 64 and the second alignment line 66 of FIG. 2, respectively. However, in other embodiments, the second and third alignment lines 106 and 108 may be presented as geometric shapes (e.g., an eyebrow and an eye, respectively) as described above with respect to FIG. 3.

Additionally, it may be desirable to position the electrode 100D on the temple 60 of the patient and approximately horizontally even with the center of the pupil 58. Accordingly, the alignment features 54 may include a fourth alignment line 110 that may be configured to approximately align with the center of the pupil 58 when the sensor 14 is applied to the patient in the desired position. As illustrated, in certain embodiments, the release liner 50 may be attached to the sensor body 40 only on portions of the sensor body 40 surrounding the electrodes 100A, 100B, and 100C. That is, the illustrated embodiment of the release liner 50 is not attached to the sensor body 40 in the area surrounding the electrode 100D. Such a configuration may facilitate the positioning of the sensor 14 on the forehead 52, because portions of the sensor 14 may be configured to curve and/or bend in order to facilitate the placement of the electrodes 100 in their respective desired positions. In this manner, the sensor 14 may be placed on the forehead 52 (e.g., or other patient anatomy) without causing the release liner 50 to bunch and/or buckle. However, it is also contemplated that the release liner 50 may be constructed such that the release liner 50 extends past the lower edge 62 of the sensor body 40, and such that the sensor 14 may be placed on the forehead 52 without causing the release liner 50 to buckle.

Further, in some embodiments, the release liner 50 may include separable portions, which may also mitigate buckling of the release liner 50. In one embodiment, the release liner 50 may include a perforated line 112 such that a first portion 114 of the release liner 50 may be removed from a second portion 116. However, any number of perforated lines is contemplated. By way of example, the caregiver may remove the first portion 114 after the electrode 100A is placed on the patient and/or may remove the second portion 116 after the electrodes 100B and 100C are placed on the patient. In other embodiments, more than one release liner 50 may be provided on the sensor body 40. That is, instead of the perforated line 112, the first and the second portions 114 and 116 may be provided as separate release liners 50.

As noted above, the alignment features 54 may include measurement lines to further facilitate the positioning of the sensor 14 relative to one or more anatomical features of the patient (e.g., the nose 104). In particular, the sensing components of the sensor 14 (e.g., the emitter 16, the detector 18, and/or the electrodes 100) may be configured for placement on the patient at a specific distance from an anatomical feature of the patient. For example, it may be desirable to position the electrode 100A on the center of the forehead 52 such that the electrode 100A is approximately two inches (five centimeters) above the bridge of the patient's nose 104. Accordingly, FIG. 5 illustrates an embodiment of the sensor 14 including the electrodes 100 and the release liner 50 having two or more measurement lines 120. The two or more measurement lines 120 may include at least a first measurement line 122 located on the release liner 50 or the sensor body 40 above (i.e., on a layer of the sensor above) the electrode 100A and a second measurement line 124 located on the release liner 50 that is approximately two inches (five centimeters) from the first measurement line 122. As illustrated, the two or more measurement lines 120 may also include a third measurement line 126 to mark the inch between the first and the second measurement lines 122 and 124. Alternatively, the two or more measurement lines 120 may include four measurement lines, or any suitable number of measurement lines, in between the first and the second measurement lines 122 and 124 to mark each centimeter. Additionally, the release liner 50 may include numerical labels 128 corresponding to each inch or centimeter. Furthermore, to facilitate the measurement from the bridge of the nose, the alignment features 54 may include parallel alignment lines 130 that may be configured to align with the bridge of the nose when the sensor 14 is applied to the forehead 52. However, in other embodiments, a geometric shape, a single line, a dot, a symbol, and/or text may be provided to identify the bridge of the nose. Furthermore, while the illustrated embodiment relates to measurement lines to facilitate the positioning of the electrode 100A of the sensor 14, it is contemplated that measurement lines may be provided to facilitate the positioning of other electrodes 100 of the sensor 14 and/or the sensing components (e.g., the one or more emitters 16 and/or the one or more detectors 18) of the sensor 14 configured to obtain pulse oximetry measurements, regional oximetry measurements, and/or any other physiological parameter measurements.

With the foregoing in mind, methods of manufacturing and methods of remanufacturing the embodiments of the sensor 14, as described above with respect to FIGS. 2-5, are also contemplated. For example, FIG. 6 illustrates an embodiment of a method 140 for manufacturing a medical sensor, such as the sensor 14. The method 140 includes providing a sensor body (e.g., the sensor body 40) (block 142). In certain embodiments, the sensor body may include one or more layers, such as structural layers (e.g., polyester, polyurethane, polypropylene, polyethylene, polyvinylchloride, acrylics, nitrile, PVC films, or acetates), foam layers (e.g., polyester foam, polyethylene foam, or polyurethane foam), adhesive layers (e.g., an acrylic-based adhesive, a supported transfer tape, an unsupported transfer tape, a hydrocolloid adhesive, or any combination thereof). For example, in certain embodiments, the sensor body may include an adhesive layer disposed on a patient-contacting surface (i.e., tissue-contacting surface) of the sensor body. The method 140 also includes providing at least one sensing element on the sensor body (block 144). For example, the at least one sensing element may include the at least one emitter 16 and the at least one detector 18, the one or more electrodes 100 (e.g., 100A, 100B, 100C, and 100D), and/or any other suitable sensing element.

Additionally, the method 140 includes providing a release liner (e.g., the release liner 50) adapted to be removably applied (e.g., removably attached) to a non-patient-contacting surface of the sensor body (block 146). As described above, the release liner may be formed from any suitable materials, such as papers, fabrics, Kraft papers, plastic films (e.g., biaxially-oriented polyethylene terephthalate, biaxially-oriented propylene, polyolefins, etc.), or the like. Furthermore, as described above, the release liner may include one or more alignment features (e.g., the alignment features 54) to facilitate the positioning of the sensor on the patient. In certain embodiments, providing the release liner may include removably applying the release liner to the top portion of the sensor body via an adhesive, such that the release liner may be pulled off of the sensor body. In other embodiments, the release liner may include perforations to facilitate the removal of the release liner from the sensor body. In this manner, the release liner may be applied to any layer of the sensor body, except for the tissue-contacting surface of the sensor body. That is, the release liner may be applied between internal layers of the sensor body, and the external portion of the release liner (e.g., the portion extending past the sensor body) may be removed using the perforations. Thus, in certain embodiments, the release liner may include portions that are non-removable from the sensor.

In some embodiments, it may be desirable to include alignment features (e.g., the alignment features 54) on a used sensor (e.g., in a remanufacturing context). Referring now to FIG. 7, an embodiment of a method 160 for remanufacturing a medical sensor, such as the sensor 14, is illustrated. The method 160 begins with obtaining a used version of the sensor 14 (block 162). The used version of the sensor 14 may be a single-use medical sensor (i.e., for use on a single patient) or may be a reusable sensor. The sensor 14 may be obtained, as an example, by a technician or similar manufacturing personnel. The sensor 14 may be sterilized before or after the acts represented by block 162 such that the sensor 14 is suitable for handling by a technician or similar worker. The sensor 14 may also undergo inspection and/or testing to determine the operability of the sensor 14 (block 164). As an example, the testing may include testing the operation and accuracy of the sensing components of the sensor 14 (e.g., the emitter 16 and the detector 18, or the electrodes 100), the sensor cable 20, and any other electronic features of the sensor 14 subject to operational degradation or failure.

After the sensor 14 has been inspected and tested, it may be determined whether it is appropriate to remanufacture the sensor 14 (query 166). For example, it may be determined whether the sensor 14 includes suitable components for remanufacture (e.g., by reviewing the results of the sensor testing acts of block 164 and/or visual inspection). Alternatively or additionally, it may be determined whether the sensor 14 has undergone previous iterations of remanufacturing. Accordingly, the sensor 14 may include one or more indications as to whether the sensor 14 has been previously remanufactured, such an external mark on the sensor 14 or a counter stored on a memory unit of the sensor 14.

In embodiments where remanufacture is not appropriate, the used version of the sensor 14 may be discarded (block 168). For example, one or more features of the used version of the sensor 14 may be inoperative, such as sensing components of the sensor 14, the cable 20, and so on. Depending on the degree to which the sensor 14 may be inoperative, it may no longer be cost-effective to remanufacture, and the sensor 14 may be discarded. In other embodiments, as mentioned above, the sensor 14 may have an external mark or a stored counter that indicates that the sensor 14 is not suitable for remanufacture.

Conversely, in embodiments where it is determined that at least a portion of the sensor 14 is suitable for remanufacturing, the sensor 14 may be prepared for remanufacturing (block 170). For example, the preparation may include cleaning the sensor 14, and may also include sterilizing the sensor 14. For example, the acts of block 170 may include sterilizing the sensor 14 using ethylene oxide (EtO) gas, gamma irradiation, autoclaving, Pasteurization, chemical antiseptics, or other such materials and methods. Sterilization may be performed at the same facility as other remanufacturing steps, or may be performed at a separate facility.

Once the sensor has been prepared for remanufacturing, the patient-contacting adhesive layer may be replaced (block 172). It may be desirable to replace the patient-contacting adhesive layer, because the adhesive may have a reduced adhesiveness due to use. Additionally, it may be desirable to replace the patient-contacting adhesive layer due to its increased level of exposure to the environment during use (e.g., to patient tissue). Specifically, the patient-contacting adhesive layer may be removed (e.g., cut, shaved, or pulled off) and a new adhesive layer may be applied. The patient-contacting adhesive layer may be replaced with the same or a different adhesive (e.g., an acrylic adhesive or a hydrocolloid layer). Additionally, in certain embodiments, one or more foam layers of the sensor 14, if present, may be replaced.

After the new patient-contacting adhesive layer is applied, a new release liner may be placed on the patient-contacting adhesive layer (block 174). The new release liner may include any liner having a release material suitable for use with the patient-contacting adhesive layer, such as a coated release paper or a release plastic film. Example release materials may include polyolefins (e.g., polypropylene, high- and low-density polyethylene), polyesters (e.g., biaxially-oriented polyethylene terephthalate), polyvinyl alcohol, Kraft paper, polystyrene or the like.

A non-patient-contacting adhesive layer may also be replaced (block 176). In particular, the non-patient-contacting adhesive layer may be an adhesive layer configured to attach the release liner 50 to the sensor body 40. As described above, the release liner 50 may be attached to any suitable layer of the sensor body 40. Accordingly, in certain embodiments, replacing the non-patient-contacting adhesive layer may include removing one or more layers of the sensor body 40. In other embodiments, the non-patient-contacting adhesive layer may be disposed on the top surface of the sensor body 40, and thus, layers of the sensor body 40 may not be removed. Specifically, the non-patient-contacting adhesive layer may be removed (e.g., cut, shaved, or pulled off) and a new adhesive layer may be applied. The non-patient-contacting adhesive layer may be replaced with the same or a different adhesive (e.g., an acrylic adhesive or a hydrocolloid layer).

After the new non-patient-contacting adhesive layer is applied, a new release liner 50 may be placed on the non-patient-contacting adhesive layer (block 178). The new release liner may include any liner having a release material suitable for use with the non-patient-contacting adhesive layer, such as a coated release paper or a release plastic film. Example release materials include polyolefins (e.g., polypropylene, high- and low-density polyethylene), polyesters (e.g., biaxially-oriented polyethylene terephthalate), polyvinyl alcohol, Kraft paper, polystyrene or the like. In accordance with present embodiments, the release liner 50 may include the one or more alignment features 54 to facilitate the placement of the sensor 14 on the patient.

After the sensor 14 has been remanufactured, the sensor 14 is then tested to ensure that it is within certain operational tolerances (block 180). For example, the sensor 14 may be attached or otherwise coupled to a test rig, which may determine and, if suitable, adjust various operational parameters of the sensor 14. The sensor 14 may then be packaged (block 182) and sent to a medical facility for use.

While the disclosure may be susceptible to various modifications and alternative forms, specific embodiments have been shown by way of example in the drawings and have been described in detail herein. However, it should be understood that the embodiments provided herein are not intended to be limited to the particular forms disclosed. Rather, the various embodiments may cover all modifications, equivalents, and alternatives falling within the spirit and scope of the disclosure as defined by the following appended claims.

What is claimed is:

1. A medical sensor, comprising:
a sensor body comprising a patient-contacting surface and a non-patient-contacting surface;
at least one sensing element disposed on or in the sensor body and configured to generate a signal representative of a physiological parameter of a patient; and
a release liner disposed on the non-patient-contacting surface of the sensor body, wherein at least a portion of the release liner extends past an edge of the sensor body and is configured to be removed from the medical sensor, and wherein the release liner comprises one or more alignment features configured to facilitate placement of the sensor at a predetermined position relative to one or more anatomical features of the patient.

2. The sensor of claim 1, wherein the one or more alignment features comprise a horizontal line, a vertical line, a geometric shape, or any combination thereof, and wherein each of the one or more alignment features is configured to approximately align with a respective anatomical feature of the patient when the sensor is applied to the patient in the desired position.

3. The sensor of claim 2, wherein each of the one or more alignment features comprises a label indicating the respective anatomical feature of the patient.

4. The sensor of claim 1, wherein the release liner is transparent or semi-transparent.

5. The sensor of claim 1, wherein the release liner comprises a perforated line along the portion of the release liner that extends past the edge of the sensor body, wherein the perforated line enables the portion of the release liner to be removed from the sensor.

6. The sensor of claim 1, wherein the at least one sensing element comprises an emitter configured to emit one or more wavelengths of light and a detector configured to detect the one or more wavelengths of light to generate the signal representative of the physiological parameter of the patient.

7. The sensor of claim 6, wherein the sensor comprises a forehead pulse oximetry sensor, and wherein the one or more alignment features comprise a first alignment line configured to approximately align with an eyebrow of the patient when the sensor is applied to the patient in the predetermined position.

8. The sensor of claim 7, wherein the one or more alignment features comprise a second alignment line or a first geometric shape that is configured to approximately align with an eye of the patient when the sensor is applied to the patient in the predetermined position.

9. The sensor of claim 1, wherein the sensor comprises a bispectral index sensor and wherein the at least one sensing element comprises one or more electrodes.

10. The sensor of claim 9, wherein the one or more alignment features comprise a first alignment line configured to approximately align with the nose of the patient when the sensor is applied to the patient in the desired position.

11. A system, comprising:
a sensor, comprising:
a sensor body comprising a patient-contacting surface and a non-patient-contacting surface;
at least one sensing element disposed on or in the sensor body and configured to generate a signal representative of a physiological parameter of a patient; and
a release liner disposed on the non-patient-contacting surface of the sensor body, wherein at least a portion of the release liner extends past an edge of the sensor body and is configured to be removed from the sensor, and wherein the release liner comprises one or more alignment features configured to facilitate placement of the sensor in a predetermined position relative to one or more anatomical features of the patient; and
a patient monitor operatively coupled to the sensor, wherein the patient monitor comprises a processor configured to receive the signal and to calculate the physiological parameter of the patient based at least in part upon the signal.

12. The system of claim 11, wherein the one or more alignment features comprise a horizontal line, a vertical line, a geometric shape, or any combination thereof, and wherein each of the one or more alignment features is configured to approximately align with a respective anatomical feature of the patient when the sensor is applied to the patient in the predetermined position.

13. The system of claim 11, wherein the sensor comprises a forehead pulse oximetry sensor, and wherein the one or more alignment features comprise a first alignment line configured to approximately align with an eyebrow of the patient when the sensor is applied to the patient in the predetermined position.

14. The system of claim 13, wherein the one or more alignment features comprise a second alignment line or a first geometric shape that is configured to approximately align with an eye of the patient when the sensor is applied to the patient in the predetermined position.

15. The system of claim 11, wherein the release liner is configured to at least partially cover an eye of the patient when the sensor is applied to the patient in the predetermined position.

16. The system of claim 11, wherein the sensor comprises a bispectral index sensor and wherein the one or more alignment features comprise a first alignment line configured to approximately align with an eyebrow of the patient and a second alignment line configured to approximately align with the nose of the patient when the sensor is applied to the patient in the predetermined position.

17. A method of manufacturing a sensor, comprising:
providing a sensor body having a patient-contacting surface and a non-patient-contacting surface;
disposing at least one sensing element on or in the sensor body, wherein the at least one sensing element is configured to generate a signal representative of a physiological parameter of a patient; and
providing a release liner adapted to be removably attached to the non-patient-contacting surface of the sensor body, and wherein the release liner comprises one or more alignment features configured to facilitate placement of the sensor at a predetermined position relative to one or more anatomical features of the patient.

18. The method of claim 11, wherein providing the release liner comprises providing a transparent or semi-transparent release liner.

19. The method of claim 11, wherein the one or more alignment features comprise a horizontal line, a vertical line, a geometric shape, or any combination thereof, and wherein each of the one or more alignment features is configured to approximately align with a respective anatomical feature of the patient when the sensor is applied to the patient in the predetermined position.

20. The method of claim 19, wherein providing the at least one sensing element comprises providing an emitter configured to emit one or more wavelengths of light and a detector configured to detect the one or more wavelengths of light, and wherein the one or more alignment features comprise an alignment line configured to approximately align with an eyebrow of the patient when the sensor is applied to the patient in the predetermined position.
